(19) United States
(12) Patent Application Publication
(10) Pub. No.: US 2024/0301620 A1
(43) Pub. Date: Sep. 12, 2024

(54) A BIO-LEATHER COATED FABRIC AND PRODUCING METHOD THEREOF

(71) Applicant: GOZEN INSTITUTE BIO MATERYAL ARASTIRMA GELISTIRME VE TASARIM ANONIM SIRKETI, ISTANBUL (TR)

(72) Inventor: Ece Gözen Akin, Bodrum/Mugla (TR)

(73) Assignee: GOZEN BIOWORKS CORP., Wilmington, DE (US)

(21) Appl. No.: 18/277,575

(22) PCT Filed: Feb. 8, 2022
(86) PCT No.: PCT/TR2022/050104

§ 371 Date: Aug. 16, 2023

(30) Foreign Application Priority Data
Feb. 19, 2021 (TR) 2021/002376

(51) Int. Cl. D06N 3/02; D06N 3/00

(52) U.S. Cl. CPC D06N 3/02; D06N 3/0006; D06N 3/0015; D06N 3/0061; D06N 3/0077; D10B 2201/02

(57) ABSTRACT

This invention relates to a bio-leather coated fabric (1) which is obtained by decomposing the cellulose layer (2) produced by microorganisms after production and adding the necessary active ingredients to obtain a biopolymer filler (3) and by pressing and laminating the obtained biopolymer filler (3) to a surface such as paper, woven fabric, reflector fabric, polyester, silk fabric in the textile industry with natural adhesives, and production method thereof (100).

### TECHNICAL FIELD

This invention relates to a bio-leather coated fabric which is obtained by decomposing the cellulose produced by microorganisms after production and adding the necessary active ingredients to obtain a biopolymer filler and by pressing and laminating the obtained biopolymer filler to a surface such as paper, woven fabric, reflector fabric, polyester, silk fabric in the textile industry with natural adhesives, and production method thereof.

### BACKGROUND ART

Coating and lamination processes are preferred in the production methods of textile products due to the appearance and performance they provide. Coating and laminated surfaces are obtained by chemical coating of one or both sides of fabrics produced as woven, knitted or non-woven surface. Chemicals used in coating are mainly chemicals such as PVC (Polyvinylchloride), PU (Polyurethane), PTFE

(Polytetrafluoroethylene, Teflon), PVDC (Polyvinylidenechloride), EVA (Ethylene vinyl acetate), Polyolefin. Especially PVC (Polyvinylchloride) used in artificial leather coating causes many health problems such as learning difficulties, immune and hormone disorders, birth defects, genetic changes. Textiles consisting of coatings with chemical materials turn into microplastics by turning into small particles in the environment over time. Synthetic clothing produced from PVC-coated synthetic fabrics is also thought to be the main source of microplastics in the air, depending on fashion, season and chemical substance. In this sense, synthetic textiles not only add microplastics to the water when washed, but also mix into the air, creating a great danger for human and environmental health indoors and outdoors.

In existing systems, toxic gases are released in production processes such as flame lamination, film lamination, pva lamination, which are widely used in the lamination process used in the textile industry, and the release of the gases to the environment threatens human and environmental health. Although coating and lamination in the production of artificial leather, which is developed as an alternative to real leather, is more environmentally friendly than and alternative to real leather, it is problematic for the environment as they do not biodegrade and take centuries to decompose in the natural environment. In this case, there is a need for artificial leathers produced entirely from waste materials or natural bacteria or fungi, and bio-leather coated fabric production methods that minimize the chemical and physical damage by obtaining the said artificial leathers with non-harmful chemical active substances and by making the fabric coating process with these bio-featured leathers.

In the state of the art, biopolymer coated fabric is described in European patent document numbered EP3239373 (A1) and priority dated 27 Apr. 2016. In this study, the application of cellulose produced using bacteria to the fabric surface was studied. Cellulose, which is indicated to be applicable to different fabric types, is dried on the fabric surface. The application is made on a single surface of the fabric and thus, an artificial leather surface is created. The process of applying the cellulose produced from bacteria on the fabric is found in the document in the state of the art. However, the invention, which is the subject of the application, is completely different from the aforementioned document in terms of finishing operation, application on glue and no waste material.

Production of a nano silver-coated bacterial cellulose fiber is disclosed in the state-of-the-art United States patent document numbered US2013211308 (A1) and priority dated 24 Apr. 2006. In this study, cellulose is produced with acetobacter-based bacteria. The cellulose produced is processed in sodium hydroxide solution. Pure cellulose was obtained after these washing processes. It has been stated that synthetic and natural polymers can also be used in different applications. In the invention, which is in the state of the art, the production of cellulose by using bacteria (acetobacter) and the process of washing the cellulose for use are carried out. However, in the invention that is the subject of the application, a biopolymer layer is produced and coated on the fabric. At the same time, the biopolymer filler obtained in the invention, which is the subject of the application, is produced by microorganisms by the fermentation method and the cellulose layer produced is cleaned beforehand and then completely disintegrated by a mechanical disintegrator. In the invention, which is the subject of the application, a biopolymer filler is obtained by adding different active ingredients (such as pectin, glycerol, tylose-c, coconut oil and indigo) into the disintegrated cellulose layer.

A glucose oxide coating production method is described in the Chinese patent document numbered CN107354144 (A) and priority dated 31 Jul. 2017, which is in the state of the art. In this study, bacteria-based cellulose produced from coconut was used. Algae culture is also added to the mixture as a vegetable mass and the resulting mixture is coated on the cotton product. The solvent in the mixture provides the necessary adhesion strength. In the aforementioned study, which is in the state of the art, bacteria-based cellulose is produced with waste materials produced from coconuts. It is completely different from the invention in the aforementioned document in terms of the process of obtaining a biopolymer filler by decomposing and purifying the biopolymer layer formed in the invention subject to the application and adding active ingredients thereto, and laminating the said filler on a fabric and drying it.

In the bio-leather coated fabric and production method thereof, which is the subject of the application, a microbial cellulose layer is first produced by microorganisms by fermentation method. The cellulose layer produced is made sterile by purifying it from microbial residues with distilled (pure) water. After sterilizing the cellulose layer, the purified layer is disintegrated using a mechanical disintegrater. Then, a biopolymer filler is obtained by adding different active ingredients (such as pectin, glycerol, tylose-c, coconut oil and indigo) into the disintegrated cellulose layer. The created biopolymer filler is laminated to the surface of the woven fabric to be coated wet and left to dry. Press application is carried out on the laminated fabric surface by means of a roller. Finally, in the invention, which is the subject of the application, the finishing process is carried out with the fabric biopolymer filler to obtain a bio-leather coated fabric.

The technical features of the invention subject to application and the technical effects provided by the invention subject to application are not found in current applications. In present embodiments, microbial cellulose layer produced by the fermentation method is purified and disintegrated and then the active substances are added to obtain a biopolymer filler. There is no method for obtaining a bio-leather coated fabric by wet coating the obtained biopolymer filler on the fabric surface, drying it, and then pressing the laminated fabric surface with a cold roller and finishing.

### Objects of Invention

The object of the present invention is to provide a bio-leather coated fabric obtained by using a bio-based alternative leather instead of real leather, and to perform the production method thereof.

Another object of the present invention is to provide a bio-leather coated fabric developed instead of PVC coated artificial leather, and to perform the production method thereof.

Another object of the present invention is to provide a bio-leather coated fabric and to perform the production method thereof that prevents climate change and environmental pollution by reducing greenhouse gas emissions associated with traditional leather and animal farms.

Another object of this invention is to provide a bio-leather coated fabric whose raw material does not contain petroleum and toxic substances, with the increase in demand for natural materials and sustainable production methods, which have changed completely with the pandemic and the global climate change crisis, and to perform the production method thereof.

Another aim of the present invention is to provide a bio-leather coated fabric that can be used as an alternative to animal hide without the need to kill any animal, that will minimize other resources such as time spent in raising animals, consumed energy and water, and that provides a “unique appearance” leather also called bio-leather, vegan leather or ecological leather to the textile and leather industry, and to perform the production method thereof.

Another aim of the present invention is to provide a bio-leather coated fabric that is environmentally friendly, of which both the materials used in production and the production method are completely sustainable, with high absorbency and high mechanical properties, which can be applied to the textile and leather industry, and to perform the production method thereof.

Another object of the present invention is to provide a bio-leather coated fabric with a tensile strength twice as high as leather, and to perform the production method thereof.

Another object of the present invention is to provide a bio-leather coated fabric that provides a “unique” coating appearance that is very similar to real leather, both physically and in appearance, and to perform the production method thereof.

### SUMMARY OF INVENTION

A bio-leather coated fabric and production method thereof, as defined in the first claim and other claims depending on this claim, realized in order to achieve the object of the present invention, provides that artificially grown leather, which is similar to real leather and does not contain chemicals, is formed and coated in a woven fabric. In the production method of the bio-leather coated fabric, firstly the woven fabric surface to be used for coating is prepared for coating. Afterwards, the biopolymer layer to be coated on the woven fabric surface is prepared. Biopolymer layer, in other words, microbial cellulose layer is produced by microorganisms by fermentation method.

The microbial cellulose layer produced by microorganisms by the fermentation method to coat the woven fabric surface is preferably prepared as follows: preparing a solution by adding black tea extract and glucose into pure water, and when the prepared solution reaches a temperature of 20-40° C., adding acetic acid and cellulose producing bacteria or cellulose producing fungi to prepare the microorganism culture by multiplying the said bacteria or fungi, and then, preparing a solution by adding 1-10% of black tea extract, 1-10% of green tea extract, and 1-10% of glucose to pure hot water, and when the prepared solution reaches the appropriate temperature, preparing fermentation liquid medium by adding 1-5% of acetic acid and glycerol and 1-10% of lichen mixture into it, producing and growing bacterial biopolymer layer by adding the prepared microorganism culture into the said fermentation liquid medium.

To obtain a cellulose layer with biopolymer properties, a solution is prepared by adding black tea extract and glucose into pure water as raw material. When the prepared solution reaches a temperature between 20-40° C., acetic acid and cellulose producing bacteria or cellulose producing bacteria or cellulose producing bacteria and fungus are added and the said bacteria or fungi are multiplied, and a multiplied microorganism culture is prepared. Afterwards, 1-10% of black tea extract, 1-10% of green tea extract and 1-10% of glucose are added into pure hot water to prepare a solution. When the prepared solution reaches the appropriate temperature, a mixture of 1-5% acetic acid and glycerol and 1-10% lichen is added into it, and the fermentation liquid medium that is required for obtaining the microbial cellulose layer is prepared. Afterwards, the microorganism culture is transferred to the growth baths in the liquid medium and it is produced by growing the pure bacterial biopolymer layer in static culture in the growth bath. Thus, microbial cellulose layer produced by microorganisms by fermentation method is obtained.

Then, the resulting cellulose layer is made sterilized by purifying it from microbial residues with distilled water. The sterilized cellulose layer is preferably disintegrated by mechanical disintegraters. In a different embodiment of the invention, the cellulose layer can be disintegrated by chemical methods. After the disintegration of the cellulose layer, biopolymer filler is formed by adding 1-10% of pectin, 1-10% of glycerol, 1-10% of Tylose-c, 1-10% of coconut oil and 1-10% of preferably natural organic dye into the disintegrated cellulose layer as active ingredients. The created biopolymer filler is laminated to the surface of the woven fabric to be coated wet and is dried. After drying the woven fabric coated with the biopolymer filler, the woven fabric surface on which the biopolymer filler is dried is passed through a cold roller to be pressed. A bio-leather coated fabric is obtained by performing the finishing process on the pressed woven fabric surface. Thus, a bio-leather coated woven fabric with completely bio-featured organic form is obtained by using residue materials and without using harmful chemicals. Said bio-leather coated woven fabric can be produced between 7 and 14 days by the methods mentioned above.

### DETAILED DESCRIPTION OF THE INVENTION

The bio-leather coated fabric and production method thereof carried out to achieve the objects of the present invention are shown in the attached figures, which are;

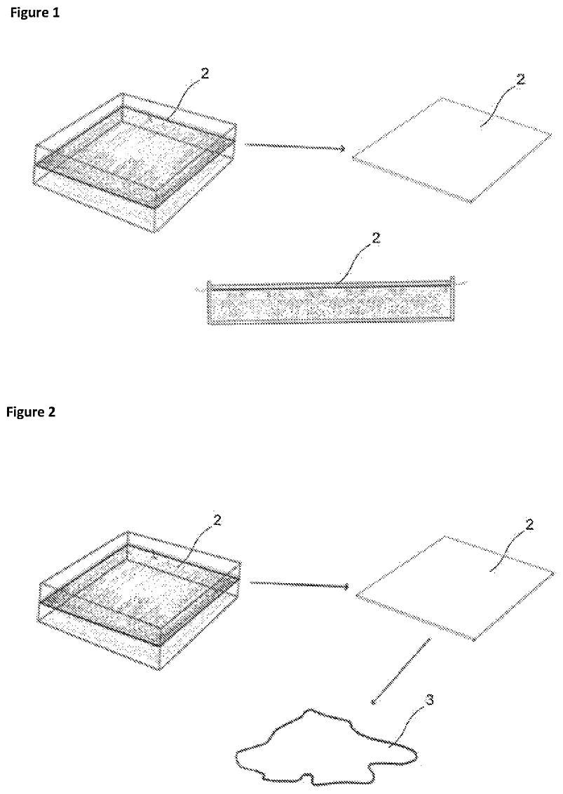

FIG. 1. It is a schematic view of microbial cellulose and production process step thereof.

FIG. 2. It is a schematic view of microbial cellulose, biopolymer filler and processing step.

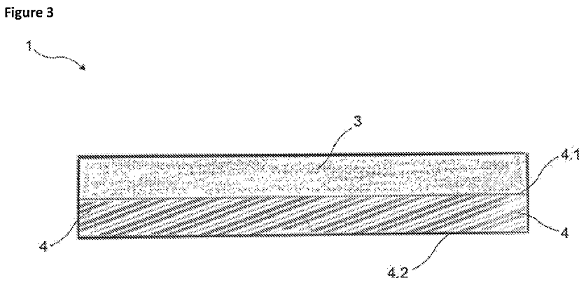

FIG. 3. It is the cross-sectional view of the bio-leather coated fabric after it is obtained.

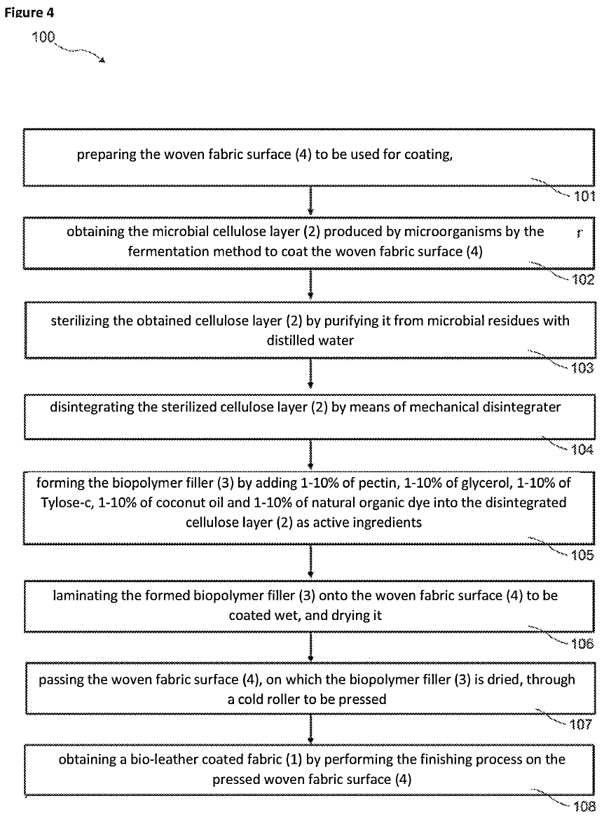

FIG. 4. It is the view of the flow chart of the bio-leather coated fabric production method.

The parts in the figures are numbered individually and the correspondence of these numbers are given below.
1. Bio-leather coated fabric
2. Cellulose layer
3. Biopolymer filler
4. Woven fabric surface
   4.1. Woven fabric front face
   4.2. Woven fabric back face
100. Production method of bio-leather coated fabric

Bio-leather coated fabric, which is similar to real leather and grown under artificial conditions without chemicals and production method thereof (100), in its most basic form, include the process steps of:
preparing (101) the woven fabric surface (4) to be used for coating,
obtaining (102) the microbial cellulose layer (2) produced by microorganisms by the fermentation method to coat the woven fabric surface (4),
sterilizing (103) the obtained cellulose layer (2) by purifying it from microbial residues with distilled water,
disintegrating (104) the sterilized cellulose layer (2) by means of mechanical disintegrater,
forming (105) the biopolymer filler (3) by adding 1-10% of pectin, 1-10% of glycerol, 1-10% of Tylose-c, 1-10% of coconut oil and 1-10% of natural organic dye into the disintegrated cellulose layer (2) as active ingredients.
laminating (106) the formed biopolymer filler (3) onto the woven fabric surface (4), preferably to be coated wet, and drying it,
passing (107) the woven fabric surface (4), on which the biopolymer filler (3) is dried, through a cold roller to be pressed,
obtaining (108) a bio-leather coated fabric (1) by performing the finishing process on the pressed woven fabric surface (4).

With the bio-leather coated fabric and production method thereof (100), which is the subject of the application, bio-leather coated fabric (1), which is similar to real leather and grown in artificial conditions without chemicals, is produced. In the bio-leather coated fabric and production method (100), a bio-based alternative fabric is developed instead of real leather. The bio-leather coated fabric and the production method thereof are developed instead of PVC coated artificial leather. The bio-leather coated fabric and its production method (100) prevent climate change and environmental pollution by reducing greenhouse gas emissions associated with traditional leather and animal farms. The bio-leather coated fabric and the production method thereof (100) do not contain petroleum and toxic substances as raw materials, with the increase in demand for natural materials and sustainable production methods, which have changed completely with the pandemic and the global climate change crisis. In the bio-leather coated fabric and the production method thereof (100), a bio-leather coated fabric (1) that can be used as an alternative to animal hide without the need to kill any animal, that will minimize other resources such as time spent in raising animals, consumed energy and water, and that provides a “unique appearance” leather also called bio-leather, vegan leather or ecological leather to the textile and leather industry can be produced. The bio-leather coated fabric and the production method thereof (100) are environmentally friendly, reveal a new structuring that can be applied in the textile and leather industry with high absorbency and high mechanical properties, where both the material itself and the production methods are completely sustainable. In the bio-leather coated fabric and its production method (100), it is provided the production of a bio-leather coated fabric (1), which is a real replacement indicator, with its tensile strength being twice as high as leather. The bio-leather coated fabric and its production method (100) provide a “unique” coating appearance of the developed bio-leather coated fabric, which is very similar to real leather, both physically and in appearance. In the bio-leather coated fabric and production method thereof (100), bio-leather coated fabric (1) is obtained, which resembles real leather both physically and in appearance, does not contain any chemical and carcinogenic substances, or the raw material of which is produced by microorganisms without the need for killing animals.

In an embodiment of the invention, the bio-leather coated fabric and the woven fabric surface (4) to be used for the coating in the production method (100) are prepared (101). In other embodiments of the invention, ready-made fabric for the coating process or a raw woven fabric woven with 100% organic cotton thread can be used as the woven fabric. In this type of fabrics, there is no need for an additional process for coating. The said woven fabric is directly ready for the coating process. After the preparation (101) of the woven fabric surface (4), the microbial cellulose layer (2) produced by the microorganisms by the fermentation method is obtained to coat the woven fabric surface (4) (102). In the process step of 102, while the cellulose layer (2) is obtained, firstly, the pre-propagation of microorganisms that can produce cellulose is carried out. In the said process step of 102, a solution is prepared by adding 1-10% of black tea extract, 1-10% of green tea extract and 1-10% of glucose to pure hot water as raw materials. The solution is preferably prepared by adding 8 grams of black tea extract and 25 grams of glucose into 1 liter of pure water. When the prepared solution reaches an appropriate temperature, 100 ml of acetic acid and cellulose producing bacteria and/or fungi are added and the said bacteria or fungi are multiplied, and a multiplied microorganism culture is prepared. Following the preparation of the said solution, 100 ml of acetic acid and cellulose producing bacteria and/or fungi are added after the temperature of the prepared solution drops to the range of 20-40° C., preferably below 30° C. Thus, a multiplied microorganism culture is prepared by multiplying the said bacteria or fungi.

In the bio-leather coated fabric production method (100) of the invention, which is the subject of the application, a stock culture is created by multiplying stock kombu moulds containing as the said microorganisms capable of producing cellulose Komagataeibacter xylinus, formerly known as Acetobacter xylinum or Gluconacetobacter xylinus and bacteria of the genus Bacterium gluconicum and yeast of the genus Saccharomyces cerevisiae and Schizosaccharomyces pombae. Multiplied microorganism culture is obtained through the stock culture created with microorganisms capable of producing cellulose. The layer forming the microbial cellulose layer (2) is selected from a sugar-based biopolymer consisting of a symbiotic culture of beneficial bacteria and yeasts, and a mixture of acetic acid bacteria. Microorganisms producing the microbial cellulose layer (2) are selected from the groups of the gram-negative aerobic bacilli of the Acetobacteraceae family, especially the genus of Komagataeibacter xylinus, formerly known as Acetobacter xylinum, or Gluconacetobacter xylinus, i.e. Bacterium gluconicum and Gluconobacter oxydans, and the yeasts Saccharomyces cerevisiae, Schizosaccharomyces pombae and a mixture thereof.

Pure bacterial biopolymer cellulose layer (2), which is the raw material of bio-leather coated woven fabric (1), in other words, pure bacterial cellulose is obtained by fermentation with the microorganism culture obtained by multiplying.

Bacterial cellulose (2), which is the raw material of the bio-leather coated woven fabric (1) of invention which is the subject of the application, is used to make edible desserts in the food industry, to create artificial tissues, to treat burns and ulcers in the medical industry, and to facilitate the absorption of creams, tonics and nail polishes in the cosmetic industry. The bacterial cellulose in question is also used in the manufacture of headphones since it transmits sound waves very well. However, since the carbon sources used in the production of bacterial cellulose such as sucrose, mannitol, glycerol, soybean extract, peptone, malt extract are very costly, its production has become extremely difficult.

In the bio-leather coated woven fabric production method (100) used in this embodiment of the invention, after the culture of multiplied microorganisms is prepared, a solution is prepared by adding 1-10% black tea extract, 1-10% green tea extract and 1-10% glucose to pure hot water. In a preferred embodiment of the invention, a solution is prepared by adding 3% of black tea extract, 2% of green tea extract and 8% of glucose to pure hot water. When the prepared solution reaches the appropriate temperature, preferably drops below 30° C., a mixture of 1-5% acetic acid and glycerol and 1-10% lichen is added into it, and the fermentation liquid medium is prepared.

While preparing the fermentation liquid medium, “Parmalia sulcata” and “Hypogymnia tubulosa” lichen species fallen from the trunk or branches of the trees and are found on it, which are found in nature as organic waste, are used as the main carbon source. In the production method of bio-leather coated fabric (100), In the process step of 102, after the temperature of the solution obtained drops below 30° C., a liquid medium is obtained by adding 2% of acetic acid, 2% of glycerol, 5-10% of Parmelia sulcata and 5-10% of Hypogymnia tubulosa lichen extract mixture into it as carbon and nitrogen sources.

Leafy lichen species called “Parmalia sulcata” used in process step of 102 of the bio-leather coated fabric production method (100) are used in folk medicine to treat severe, acute colitis and gastrointestinal system diseases. The drugs produced from the extracts of the leafy lichen species called “Parmalia sulcata” have a restorative and calming effect on the mucous membrane of the respiratory tract. “Hypogymnia tubulosa” lichen species are known to have antimicrobial effects against bacteria such as E. Coli.

By using the lichens used in the process step of 102 of the bio-leather coated fabric production method (100) as the raw material of the bacterial cellulose layer (2), natural wastes are transformed into wealth and at the same time, the damage caused by the leather and artificial leather industry to the environment is tried to be minimized by the fermentation production method. With the fermentation liquid medium created in the process step of 102, an industrial production method with a very low carbon emission, a production process that is highly supportive of the green economy and the prevention of climate change emerges.

The culture medium formed during the transfer of microorganism culture into the liquid medium preferably has a temperature of 30 degrees and the pH value is between 5 and 7. The growth baths are sterilized with ethanol. In the growth baths, the necessary culture medium is created by combining the obtained liquid medium and the culture of the multiplied microorganisms. The culture medium is formed between 7 and 10 days. The temperature of the culture medium is preferably around 30° C. The pH value of the culture medium is between 5 and 7. In the bio-leather coated fabric production method (100), the cellulose layer (2) obtained after the production of the cellulose layer (2) with biopolymer properties in the growth bath is washed and purified, and a sterile biopolymer layer is obtained. The cellulose layer (2) with biopolymer properties is cleaned from all culture media and microbial residues while being washed. A sterilized biopolymer layer is obtained by washing the cellulose layer (2) with biopolymer properties in hot water at 70° C., then in cold water and finally with distilled water (103).

In the invention that is the subject of the application, the cellulose layer (2) obtained in the bio-leather coated fabric method (100) is sterilized by purifying it from microbial residues with distilled water (103). In the process step of 103, in order to clean the resulting cellulose layer (2) from microbial residues, it is obtained as purified by washing firstly in 70° C. hot water, then in cold water and finally with distilled water. The sterilized cellulose layer (2) is disintegrated by mechanical disintegranters (104). The biopolymer filler (3) is formed (105) by adding 1-10% of pectin, 1-10% of glycerol, 1-10% of Tylose-c, 1-10% of coconut oil and 1-10% of natural organic dye into the disintegrated cellulose layer (2) as active ingredients. The cellulose that consists the biopolymer filler (3) is microbial cellulose. It is also called bacterial biopolymer produced by microorganisms. Biopolymers are environmentally friendly polymers synthesized by living organisms, mostly produced from biodegradable, non-toxic and edible resources.

The biopolymer filler (3) formed in the invention that is the subject of the application, in the bio-leather coated fabric method (100) is laminated to the surface of the woven fabric (4) to be coated wet and is dried (106). In the process step of 106, while the biopolymer filler (3) is only in wet form, it is coated on the front surface (4.1) of the woven fabric (4) also in wet form by means of a single natural adhesive such as gum arabic on it as an adhesive. After the biopolymer filler (3) is dried on the woven fabric surface (4) (106), the woven fabric surface (4) on which the biopolymer filler (3) is dried is passed through a cold roller (107) to be pressed. In the process step of 107, the laminated surface is allowed to dry at room temperature for 1-2 days and a cold roller is passed over the dried surface. After then, a bio-leather coated fabric (1) is obtained by performing the finishing process on the pressed woven fabric surface (4) (108).

The finishing process is, in a sense, the make-up process of the leather. An unfinished leather is not fully finished. It is known that the finishing process is performed in order to protect the surface of the leather products found even in the findings in the ancient times and to make them look beautiful. The finishing process changes according to the developments and demands in fashion today. Desired patterns, colors, fastness properties and appearance may change every year. In addition, since the desired properties are different from leather products used in the areas such as garments, shoes, upholstery, saddlery, etc., the finishing process is performed differently according to these areas. Finishing layers are applied on the leather not as a single layer, but with multiple layers. Intense finishing mixtures and roll coat machines are preferred in order to cover the defects in leathers with damaged surfaces, while the finishes that intend not to lose the natural skin appearance of the leather, such as napa, are sprayed in thin coats in airbrush machines. As a result of the finishing process, the binder mixtures should be well bonded to the leather surface, but there should be no cracks or peeling on the floors.

On the other hand, it is necessary that the water vapor and air permeability properties of natural leather, which are superior to artificial leathers, should not be reduced excessively by finishing layers.

The bio-leather coated fabric (1), which is finalized with the finishing process in the bio-leather coated fabric method (100) in an embodiment of the invention, has such properties after being tanned with 3% of coconut oil, 3% of glycerol as being able to be sewn, being easily shaped by hand, flexibility, easy to iron, interlining, high absorbency, high water holding capacity, biocompatibility, high tensile strength and mechanical strength. The bio-leather coated fabric (1) is similar to real leather in appearance and physical structure on the front surface (4.1), while the back surface (4.2) has the appearance of a woven fabric.

In alternative embodiments of the invention, instead of the woven fabric (4), which is the surface on which the biopolymer filler (3) is coated, it can be used on acid-free handmade woven Nepalese paper and paper-like surfaces obtained from the dot plant.

The bio-leather coated fabric production method (100) in this embodiment of the invention is carried out as follows. A bio-leather coated fabric production method (100) provides that artificially grown leather, which is similar to real leather and does not contain chemicals, is formed and coated in a woven fabric. In the production method of the bio-leather coated fabric (100), firstly the woven fabric surface to be used for coating is prepared for coating (101). After the process step of 101, the microbial cellulose layer (2) produced by the microorganisms by the fermentation method is obtained to coat the woven fabric surface (4) (102). In process step of 102, to obtain a cellulose layer (2) with biopolymer properties, a solution is prepared by adding black tea extract and glucose into pure water as raw material. When the prepared solution reaches a temperature between 20-40° C., acetic acid and cellulose producing bacteria or cellulose producing bacteria or cellulose producing bacteria and fungus are added and the said bacteria or fungi are multiplied, and a multiplied microorganism culture is prepared. Then, a solution is prepared by adding 1-10% of black tea extract, 1-10% of green tea extract and 1-10% of glucose to pure hot water and when the prepared solution reaches the appropriate temperature, a mixture of 1-5% acetic acid and glycerol and 1-10% lichen is added into it, and the fermentation liquid medium that is required for obtaining the microbial cellulose layer is prepared. Afterwards, the microorganism culture is transferred to the growth baths in the liquid medium and it is produced by growing the pure bacterial biopolymer layer in static culture in the growth bath. Thus, microbial cellulose layer (2) produced by microorganisms by fermentation method is obtained.

Then, in the bio-leather coated woven fabric production method (100), the resulting cellulose layer (2) is made sterilized by purifying it from microbial residues with distilled water (103). After the process step of 103, the sterilized cellulose layer is disintegrated by mechanical disintegraters (104). After the disintegration of the cellulose layer (2), biopolymer filler (3) is formed by adding 1-10% of pectin, 1-10% of glycerol, 1-10% of Tylose-c, 1-10% of coconut oil and 1-10% of natural organic dye into the disintegrated cellulose layer (2) as active ingredients (105). The created biopolymer filler (3) is laminated to the surface of the woven fabric (4) to be coated wet and is dried (106). After drying the woven fabric coated with the biopolymer filler (3), the woven fabric surface (4) on which the biopolymer filler (3) is dried is passed through a cold roller to be pressed (107). A bio-leather coated fabric (1) is obtained by performing the finishing process on the pressed woven fabric surface (4) (108). Thus, a bio-leather coated woven fabric (1) with completely bio-featured organic form is obtained by using residue materials and without using harmful chemicals. The said bio-leather coated woven fabric (1) can be produced between 7 and 14 days by the aforementioned bio-leather coated fabric production method (100).

1. The production method (100) of bio-leather coated fabric, which is similar to real leather and grown under artificial conditions without chemicals, characterized in that it includes in its most basic form the process steps of:
preparing the woven fabric surface (4) to be used for coating, or using ready-made woven fabric (101),
obtaining (102) the microbial cellulose layer (2) produced by microorganisms by the fermentation method to coat the woven fabric surface (4), as follows:
preparing a solution by adding black tea extract and glucose into pure water, and when the prepared solution reaches a temperature of 20-40° C., adding acetic acid and cellulose producing bacteria or cellulose producing fungi to prepare the microorganism culture by multiplying the said bacteria or fungi,
preparing a solution by adding 1-10% of black tea extract, 1-10% of green tea extract, and 1-10% of glucose to pure hot water, and when the prepared solution reaches the appropriate temperature, preparing fermentation liquid medium by adding 1-5% of acetic acid and glycerol and 1-10% of lichen mixture into it,
producing and growing microbial cellulose layer (2) by adding the prepared microorganism culture into the said fermentation liquid medium,
sterilizing (103) the obtained cellulose layer (2) by purifying it from microbial residues with distilled water,
disintegrating (104) the sterilized cellulose layer (2) by means of mechanical disintegrater,
forming (105) the biopolymer filler (3) by adding 1-10% of pectin, 1-10% of glycerol, 1-10% of Tylose-c, 1-10% of coconut oil and 1-10% of natural organic dye into the disintegrated cellulose layer (2) as active ingredients.
drying (106) the formed biopolymer filler (3) by laminating it onto the woven fabric surface (4), to be coated wet,
passing the woven fabric surface (4), on which the biopolymer filler (3) is dried, through a cold roller (107) to be pressed,
obtaining (108) a bio-leather coated fabric (1) by performing the finishing process on the pressed woven fabric surface (4) (108).

2. The production method (100) of bio-leather coated fabric as in claim 1, characterized in that in the preparation (101) process step of the woven fabric surface (4) to be used for coating, woven fabric ready for coating or raw woven fabric woven with cotton thread of 100% organic structure is used as woven fabric.

3. The production method (100) of bio-leather coated fabric as in claim 1, characterized in that in the process step (102) of obtaining the microbial cellulose layer (2) produced by the microorganisms by the fermentation method to coat the woven fabric surface (4), the pre-propagation of the microorganisms capable of producing cellulose is first performed while the cellulose layer (2) is obtained.

4. The production method (100) of bio-leather coated fabric as in claim 1, characterized in that in the process step (102) of obtaining the microbial cellulose layer (2) produced by the microorganisms by the fermentation method to coat the woven fabric surface (4), a solution is prepared by adding as raw materials 8 grams of black tea extract and 25 grams of glucose into 1 liter of pure water.

5. The production method (100) of bio-leather coated fabric as in claim 1, characterized in that in the process step (102) of obtaining the microbial cellulose layer (2) produced by the microorganisms by the fermentation method to coat the woven fabric surface (4), when the prepared solution reaches the appropriate temperature, 100 ml of acetic acid and cellulose-producing bacteria and/or fungi are added and a multiplied microorganism culture is prepared by multiplying the said bacteria or fungi.

6. The production method (100) of bio-leather coated fabric as in claim 1, characterized in that in the process step (102) of obtaining the microbial cellulose layer (2) produced by the microorganisms by the fermentation method to coat the woven fabric surface (4), after the temperature of the prepared solution drops to the range of 20-40° C., preferably below 30° C., 100 ml of acetic acid and cellulose producing bacteria and/or fungi are added.

7. The production method (100) of bio-leather coated fabric as in claim 1, characterized in that in the process step (102) of obtaining the microbial cellulose layer (2) produced by the microorganisms by the fermentation method to coat the woven fabric surface (4), a culture of a multiplied microorganism is prepared by multiplying bacteria or fungi.

8. The production method (100) of bio-leather coated fabric as in claim 1, characterized in that in the process step (102) of obtaining the microbial cellulose layer (2) produced by the microorganisms by the fermentation method to coat the woven fabric surface (4), a stock culture is created by multiplying stock kombu moulds containing as the said microorganisms capable of producing cellulose Komagataeibacter xylinus, formerly known as Acetobacter xylinum or Gluconacetobacter xylinus and bacteria of the genus Bacterium gluconicum and yeast of the genus Saccharomyces cerevisiae and Schizosaccharomyces pombae.

9. The production method (100) of bio-leather coated fabric as in claim 1, characterized in that in the process step (102) of obtaining the microbial cellulose layer (2) produced by the microorganisms by the fermentation method to coat the woven fabric surface (4), multiplied microorganism culture is obtained through the stock culture created with microorganisms capable of producing cellulose.

10. The production method (100) of bio-leather coated fabric as in claim 1, characterized in that in the process step (102) of obtaining the microbial cellulose layer (2) produced by the microorganisms by the fermentation method to coat the woven fabric surface (4), the microbial cellulose layer (2) is selected from a sugar-based biopolymer consisting of a symbiotic culture of beneficial bacteria and yeasts and a mixture of acetic acid bacteria.

11. The production method (100) of bio-leather coated fabric as in claim 1, characterized in that in the process step (102) of obtaining the microbial cellulose layer (2) produced by the microorganisms by the fermentation method to coat the woven fabric surface (4), microorganisms producing the microbial cellulose layer (2) are selected from the groups of the gram-negative aerobic bacilli of the Acetobacteraceae family, especially the genus of Komagataeibacter xylinus, formerly known as Acetobacter xylinum, or Gluconacetobacter xylinus, i.e. Bacterium gluconicum and Gluconobacter oxydans, and the yeasts Saccharomyces cerevisiae, Schizosaccharomyces pombae and a mixture thereof.

12. The production method (100) of bio-leather coated fabric as in claim 1, characterized in that in the process step (102) of obtaining the microbial cellulose layer (2) produced by the microorganisms by the fermentation method to coat the woven fabric surface (4), after the multiplied culture of microorganisms is prepared, a solution is prepared by adding 1-10% of black tea extract, 1-10% of green tea extract, and 1-10% of glucose to pure hot water.

13. The production method (100) of bio-leather coated fabric as in claim 1, characterized in that in the process step (102) of obtaining the microbial cellulose layer (2) produced by the microorganisms by the fermentation method to coat the woven fabric surface (4), a solution is prepared by adding 3% of black tea extract, 2% of green tea extract, and 8% of glucose to pure hot water.

14. The production method (100) of bio-leather coated fabric as in claim 1, characterized in that in the process step (102) of obtaining the microbial cellulose layer (2) produced by the microorganisms by the fermentation method to coat the woven fabric surface (4), when the temperature of the prepared solution drops below 30° C., the fermentation liquid medium is prepared by adding 1-5% or acetic acid and glycerol and 1-10% or lichen mixture into it.

15. The production method (100) of bio-leather coated fabric as in claim 1, characterized in that in the process step (102) of obtaining the microbial cellulose layer (2) produced by the microorganisms by the fermentation method to coat the woven fabric surface (4), while preparing the fermentation liquid medium, “Parmalia sulcata” and “Hypogymnia tubulosa” lichen species fallen from the trunk or branches of the trees and are found on it, which are found in nature as organic waste, are used as the main carbon source.

16. The production method (100) of bio-leather coated fabric as in claim 1, characterized in that in the process step (102) of obtaining the microbial cellulose layer (2) produced by the microorganisms by the fermentation method to coat the woven fabric surface (4), when the temperature of the prepared solution drops below 30° C., liquid medium is obtained by adding 2% of acetic acid, 2% of glycerol, 5-10% of Parmelia sulcata and 5-10% of Hypogymnia tubulosa lichen extract mixture into it as carbon and nitrogen sources.

17. The production method (100) of bio-leather coated fabric as in claim 1, characterized in that in the process step (102) of obtaining the microbial cellulose layer (2) produced by the microorganisms by the fermentation method to coat the woven fabric surface (4), after the preparation of the fermentation liquid medium, the microorganism culture is transferred to the growth baths into the liquid medium.

18. The production method (100) of bio-leather coated fabric as in claim 1, characterized in that in the process step (102) of obtaining the microbial cellulose layer (2) produced by the microorganisms by the fermentation method to coat the woven fabric surface (4), the culture medium formed during the transfer of the microorganism culture into the liquid medium has a temperature of 30 degrees and the pH value is between 5 and 7.

19. The production method (100) of bio-leather coated fabric as in claim 1, characterized in that in the process step (102) of obtaining the microbial cellulose layer (2) produced by the microorganisms by the fermentation method to coat the woven fabric surface (4), in the growth baths, the required culture medium is created by combining the obtained liquid medium and the culture of the multiplied microorganisms.

20. The production method (100) of bio-leather coated fabric as in claim 1, characterized in that in the process step (102) of obtaining the microbial cellulose layer (2) produced by the microorganisms by the fermentation method to coat the woven fabric surface (4), culture medium is formed between 7 and 10 days.

21. The production method (100) of bio-leather coated fabric as in claim 1, characterized in that in the process step (102) of obtaining the microbial cellulose layer (2) produced by the microorganisms by the fermentation method to coat the woven fabric surface (4), the temperature of the culture medium is around 30° C., the pH value is between 5 and 7.

22. The production method (100) of bio-leather coated fabric as in claim 1, characterized in that in the process step (103) of sterilizing the obtained cellulose layer (2) by purifying it from microbial residues with distilled water, a sterilized biopolymer layer is obtained by washing the cellulose layer (2) with biopolymer properties in hot water at 70° C., then in cold water and finally with distilled water.

23. The production method (100) of bio-leather coated fabric as in claim 1, characterized in that in the process step (106) of laminating the biopolymer filler (3) onto the woven fabric surface (4) to be coated wet and drying it, while the biopolymer filler (3) is only in wet form, it is coated on the front surface (4.1) of the woven fabric (4) also in wet form by means of a single natural adhesive such as gum arabic on it as an adhesive.
